SEQUENCE LISTING (2 sequences)

SEQ ID NO: 1 - DNA, 960 bp, Bacterial phytochelatin synthetase
gtgaatttacacatatttaaacgcagtattctaaccatacacttggtaattttagctgggttaattagtggctgttacggcatttcaaataccaaagcagatactagtcaatcaatgccgaaccacaaaaacgccactcaacaaaccgcatcaggcgacgccgcgccattcgacaatggccgcatgtgtattgaccatttagttaatgccaagctaaatcactttaacgacgactggtcactacctgaaaacatggtggatttgcgcagcgaagagggccaaacccgactattagaatgcagctactgtgaagactaccccacgcttgcaaccgcctaccttactcaagccacgcaaaccttttgcggtttagcctctaccactatggtacttaatgccgatgcagaatcgagaaaatggcgcccggtgacccaaccctaccaaccttttaatttttatacacaatgtaatttattaaatgatgaagtaagggaacatatagatgttgcgaaagtacttaacgaagggatggagctaaacgaaatattttttgtggttaaaaatcagccctcggtagttgaagcctattgccaacacgcaaccagcgcgcaaaacaaaggcgaactacctgcaagtgatgcaccgcacaattgcggcgtagaccaagattacaacacttttatgaaaaccattaccaacgcgctgaacacaccaaggcactatgtaatagctaatttcgccggggctccttcgccagaacgcggcggacacttctcgccaatcgcggcctaccacaaagccagcgacagttttcttattatggatgttgctcgctataaatacccaccgttctggataagctccaaagaactttggcaagcaatgcaaaaaatagatagcggctcaggcaaagcgcgcggctatattattgctcagactaaatctggctccgccaattaa

SEQ ID NO: 2 - protein, 319 aa, Bacterial phytochelatin synthetase
MNLHIFKRSILTIHLVILAGLISGCYGISNTKADTSQSMPNHKNATQQTASGDAAPFDNGRMCIDHLVNAKLNHFNDDWSLPENMVDLRSEEGQTRLLECSYCEDYPTLATAYLTQATQTFCGLASTTMVLNADAESRKWRPVTQPYQPFNFYTQCNLLNDEVREHIDVAKVLNEGMELNEIFFVVKNQPSVVEAYCQHATSAQNKGELPASDAPHNCGVDQDYNTFMKTITNALNTPRHYVIANFAGAPSPERGGHFSPIAAYHKASDSFLIMDVARYKYPPFWISSKELWQAMQKIDSGSGKARGYIIAQTKSGSAN

(19) United States
(12) Patent Application Publication
(10) Pub. No.: US 2005/0136426 A1
(43) Pub. Date: Jun. 23, 2005

(54) Bacterial phytochelatin synthetase

(76) Inventors: Michael Howard, Annapolis, MD (US); Steven Wayne Hutcheson, Columbia, MD (US); Ronald M. Weiner, Potomac, MD (US)

Correspondence Address: ARENT FOX PLLC, 1050 CONNECTICUT AVENUE, N.W., SUITE 400, WASHINGTON, DC, 20036, US

(21) Appl. No.: 10/875,221

(22) Filed: Jun. 25, 2004

Related U.S. Application Data
(60) Provisional application No. 60/483,134, filed on Jun. 27, 2003.

(51) Int. Cl. C12Q 1/68; C07H 21/04; C12N 9/10; C12N 15/74

(52) U.S. Cl. 435/6; 435/69.3; 435/320.1; 435/193; 435/252.33; 536/23.2

(57) ABSTRACT

The present invention relates to the discovery of a gene in a bacterium expressing a protein that has an activity similar to plant phytochelatin synthetase. This gene was discovered in the marine γ-proteobacterium Microbulbifer degradans strain 2-40, and is cloned and expressed in the bacterium Escherichia coli. Bacteria expressing this gene are useful in remediation of contaminated solids and liquids and in the extraction of rare and/or valuable metals.

### CROSS-REFERENCE TO RELATED APPLICATIONS

This claims priority to U.S. Provisional Application No. 60/483,134, filed Jun. 27, 2003, the contents of which are incorporated herein, in their entirety, by reference.

### BACKGROUND OF THE INVENTION

1. Field of the Invention

The invention is generally directed to enzymes and proteins that bind, chelate, and/or sequester metals.

2. Background of the Invention

Phytochelatin synthetases are enzymes that catalyze the production of phytochelatin. Phytochelatin is composed of repeating units of glutathione, a naturally occurring peptide in many organisms. Phytochelatin synthetases polymerize the amino acid dimer to form a chain of approximately 8 to 10 glutathione units.

Phytochelatin is often used by plants to chelate heavy metals that have been delivered to their cells from root systems. After phytochelatin is saturated in planta, it is transported to a vacuole for storage. A large majority of phytochealtin synthetases are of plant origin. Plants are often challenged with toxic heavy metals that are taken up by root systems that encounter these materials in soil.

Pollution of marine environments is a serious and growing problem. Agricultural and urban runoff, sewage, and industrial waste are some of the major sources of marine pollution. These sources carry nutrients, sediments, pathogens, and toxic contaminants into coastal waters. The pollutants cause, or contribute to, “dead zones” where depleted oxygen levels in the water make it difficult for marine life to survive. In addition, over 50% of the coastal waters (e.g., estuaries, bays, harbors) of the 48 contiguous states were under fish and/or shellfish consumption advisories due to high concentrations of chemical contaminants such as mercury, PCBs or dioxin.

Some plant species have been engineered to over produce phytochelatin synthetases and are being used to remediate heavy metal contaminated soil. For example, the phytochelatin synthetase of Arabidopsis was cloned and over-expressed in E. coli. When grown in the presence of heavy metals, the recombinant E. coli was shown to sequester heavy metals. The phytochelatin synthetase genes from several plant species have been over-expressed in E. coli strains and shown to function in vivo as chelators of heavy metal from contaminated liquids.

While phytochelatin synthetases from plant sources are plentiful, there have not been any reports of a phytochelatin synthetase of bacterial origin. Proteins of bacterial origin may be more efficiently expressed in a bacterial host. Therefore, there exists a need to identify bacterial proteins and enzyme systems that behave similar to phytochelatin synthetase, to clone these genes into other suitable bacteria, and to express these genes using suitable vectors. These gene products, as well as organisms containing these genes, can be used to sequester metals for uses such as remediation and extraction.

### SUMMARY OF THE INVENTION

One aspect of the present invention is directed to proteins and enzymes that can bind to metals.

A further aspect of the invention is directed to a identifying and isolating genes and polypeptides that exhibit an activity similar to phytochelatin synthetase.

Another aspect of the present invention is directed to identifying and isolating genes and polypeptides that exhibit an activity similar to phytochelatin synthetase in bacteria and expressing these genes in other bacteria.

Another aspect of the present invention is directed to using organisms that are able to express phytochelatin synthetase to isolate and sequester metals.

A further aspect of the invention is directed to isolation and extraction of metals, including rare, valuable, and heavy metals, using bacteria that express a protein comprising an activity similar to phytochelatin synthetase.

Another aspect of the invention is directed to a method for remediating an area that contains one or more metals. The method involves (a) treating the area with a bacterium expressing phytochelatin synthetase activity; (b) allowing the bacterium to sequester the metal; (c) isolating the bacterium from the area, and (d) optionally isolating the metal from the bacterium.

Another aspect of the invention is directed to a method for identifying a nucleotide sequence encoding a polypeptide having phytochelatin synthetase activity from M. degradans. The method comprises constructing an M. degradans genomic library in E. coli and screening the library for phytochelatin synthetase activity.

A further aspect of the invention is directed to an apparatus for the extraction of a metal, comprising: (a) a comprising an organism able to express phytochelatin synthetase; (b) an inlet for a composition comprising the metal; and (c) an outlet for a composition not comprising the metal.

Other aspects, features, and advantages of the invention will become apparent from the following detailed description.

### DETAILED DESCRIPTION

Microbulbifer degradans strain 2-40 is a marine γ-proteobacterium that was isolated from decaying Sparina alterniflora, a salt marsh cord grass in the Chesapeake Bay watershed. Consistent with its isolation from decaying plant matter, M. degradans strain 2-40 is able to degrade many complex polysaccharides, including cellulose, pectin, xylan, and chitin, which are common components of the cell walls of higher plants. M. degradans strain 2-40 is also able to depolymerize algal cell wall components, such as agar, agarose, and laminarin, as well as protein, starch, pullulan, and alginic acid. In addition to degrading this plethora of polymers, M. degradans strain 2-40 can utilize each of the polysaccharides as the sole carbon source.

The present invention describes discovery of a gene from M. degradans that encodes a protein with a similarity to plant phytochelatin synthetase. While examining the genome sequence of M. degradans strain 2-40, the inventors discovered a gene that had some similarity to phytochelatin synthetases from plant sources. The corresponding protein was only 51% similar to a phytochelatin synthetase from the soybean Glycine max. This gene was isolated, cloned into E. coli and expressed. The protein is not toxic to the E. coli host, nor was it extensively degraded during expression. This protein is well tolerated in E. coli, a feature that is not discernable from protein or nucleotide sequence analysis with other (i.e., plant) phytochelatin synthetases.

Because the Microbulbifer phytochelatin is of bacterial origin, it is expressed at higher levels and is less toxic to bacterial hosts such as E. coli. In addition, the Microbulbifer phytochelatin synthetase has evolved to function in a bacterium that exists in an aqueous habitat. Plant phytochelatins have evolved to function with heavy metals delivered from root systems and may have very different optimal conditions than those observed in a bacterial cell. For example, bacterial phytochelatin synthetases may have evolved to function within bacterial cytoplasm or to sequester metals in an open environment such as the cytoplasm as opposed to a plant vacuole.

Phytochelatin synthetase protein from M. degradans is better suited for function in E. coli over similar proteins from plants. One advantage of using M. degradans phytochelatin synthetase genes in E. coli is that it does not contain exons, unlike plant phytochelatin synthetase genes. Furthermore, codon usage between bacteria is not drastic different, as it can between bacteria and plants. Finally, bacterial proteins are less toxic to bacteria than eukaryotic proteins produced by heterologous bacteria.

bpsA (SEQ ID NO: 1) is 957 bases long with GTG start and TAA stop. It codes for BpsA (SEQ ID NO: 2), which is a 319 amino-acid protein with size of about 35,000 daltons. BpsA has an unknown domain at the amino terminus and phytochelain synthetase domain at carboxy terminus

It is one aspect of the present invention to provide a nucleotide sequence that has a homology selected from 100%, 99%, 98%, 97%, 96%, 95%, 90%, 85%, 80%, or 75% to SEQ ID NO:1 with phytochelatin synthetase activity in bacteria. The present invention also covers replacement of between 1 and 20 nucleotides of SEQ ID NO:1 with non-natural or non-standard nucleotides for example phosphorothioate, deoxyinosine, deoxyuridine, isocytosine, isoguanosine, ribonucleic acids including 2-O-methyl, and replacement of the phosphodiester backbone with, for example, alkyl chains, aryl groups, and protein nucleic acid (PNA).

It is another aspect of the present invention to provide a nucleotide sequence that hybridizes to SEQ ID NO:1 under a stringency condition of 1×SSC. It is another aspect of the present invention to provide a nucleotide sequence that hybridizes to SEQ ID NO:1 under a stringency condition of 2×SSC. It is another aspect of the present invention to provide a nucleotide sequence that hybridizes to SEQ ID NO:1 under a stringency condition of any one of 3×SSC. It is another aspect of the present invention to provide a nucleotide sequence that hybridizes to SEQ ID NO:1 under a stringency condition of 4×SSC. It is another aspect of the present invention to provide a nucleotide sequence that hybridizes to SEQ ID NO:1 under a stringency condition of 5×SSC. It is another aspect of the present invention to provide a nucleotide sequence that hybridizes to SEQ ID NO:1 under a stringency condition of 6×SSC. It is another aspect of the present invention to provide a nucleotide sequence that hybridizes to SEQ ID NO:1 under a stringency condition of 7×SSC. It is another aspect of the present invention to provide a nucleotide sequence that hybridizes to SEQ ID NO:1 under a stringency condition of 8×SSC. It is another aspect of the present invention to provide a nucleotide sequence that hybridizes to SEQ ID NO:1 under a stringency condition of 9×SSC. It is another aspect of the present invention to provide a nucleotide sequence that hybridizes to any one of SEQ ID NO:1 under a stringency condition of 10×SSC.

It is another aspect of the present invention to provide a nucleotide sequence that encodes a polypeptide comprising phytochelatin synthetase activity in bacteria. It is well understood that due to the degeneracy of the genetic code, an amino acid can be coded for by more than one codon. Therefore, the present invention encompasses all polynucleotides that code for SEQ ID NO: 2.

The scope of this invention covers natural and non-natural alleles of SEQ ID NO: 2. In a preferred embodiment of the present invention, alleles of SEQ ID NO: 2 comprise replacement of one, two, three, four, or five naturally occurring amino acids with similarly charged, shaped, sized, or situated amino acids (conservative substitutions). The present invention also covers non-natural or non-standard amino acids for example selenocysteine, pyrrolysine, 4-hydroxyproline, 5-hydroxylysine, phosphoserine, phosphotyrosine, and the D-isomers of the 20 standard amino acids.

Phytochelatin synthetase enzymes that can be expressed in bacteria have a number of uses. One embodiment of the present invention comprises bacteria that are able to express a gene similar to phytochelatin synthetase, which are cultured in liquids containing quantities of metals, metalloids, or non-metals of interest. The metals, metalloids, or non-metals of interest can exist as ionic, simple or complex, or non-ionic species. These bacteria sequester and concentrate these metallic elements (including metalloids). The metals can then be extracted from the bacteria. In theory any metal can be sequestered, concentrated, and extracted by this process. Examples of metals and metalloids include Ag, Au, Ba, Bi, Cd, Co, Cs, Cu, Dy, Er, Eu, Fe, Ga, Gd, Hg, Ho, In, Ir, La, Lu, Mn, Mo, Nb, Ni, Os, Pt, Pb, Pd, Rb, Rh, Sc, Se, Sn, Ta, Ti, TI, U, V, W, Y, Yb, Zn, and Zr.

Another embodiment of the present invention comprises bacteria that are able to express a gene similar to phytochelatin synthetase, which are used to chelate heavy metal waste from a variety of industrial wastewaters and liquids. Many bacteria that are resistant to heavy metals are able to survive because of the presence of membrane pumps that continuously pump heavy metal ions back into the environment. This has no value to bioremediatory efforts. Bacteria that are able to code for phytochelatin synthetase and able to tolerate heavy metal ions allow chelation and sequestering of heavy metals, thus removing them from the environment. The bacteria containing the metals are isolated. Most of the heavy metals would then be sequestered in a much smaller and more concentrated volume (e.g., the bacterial pellet), permitting facile disposal or release of the liquid portion of the reaction.

In a preferred embodiment, metals dissolved in a large liquid volume can be collected in a single step, for example using bioreactors. These bioreactors containing bacteria that express the phytochelatin genes are fed with metal-containing liquids and the metals are extracted. These bioreactors can be of any type including batch-type, continuous flow, and plug-flow.

Non-limiting examples of experimental methods used in the present invention are described.

Growth of bacterial strains. M. degradans strain 2-40 was grown in minimal medium containing (per liter): 2.3% Instant Ocean, 0.5% ammonium chloride, 0.2% glucose, and 50 mM Tris HCl, pH 7.6. Other carbon sources were added to a final concentration of 0.1%. Agar was added to a final concentration of 1.5% to prepare solid media. All cultures were incubated at 25° C. E. coli EC300, DH5αE, and Tuner strains were grown in Luria-Bertani (LB) broth or agar supplemented with the appropriate antibiotics and incubated at 37° C.

Construction of an M. degradans strain 2-40 genomic library. Strain 2-40 chromosomal DNA was isolated and prepared for ligation into pCC1. Sau3A fragments of 30 to 40 kb were isolated using gel extraction and ligated into Bam H1-digested pCC1. The vector was packaged into phage and used to infect E. coli EC300. Transductants were selected using chloramphenicol (30 μg/mL).

Screening of the M. degradans strain 2-40 genomic library for phytochelatin synthetase activity. M. degradans and E. coli transductants could be screened for phytochelatin synthetase activity by growing them in the presence of heavy metals in liquid medium. After the cells have grown to a density suitable for collection, they can be collected, dried, and mineralized. If the cells are expressing phytochelatin synthetase and are chelating the metals, the amount of heavy metals in M. degradans or E. coli expressing the protein should be much higher than M. degradans and E. coli not expressing the protein. Phytochelatin synthetase activity can be identified by atomic absorption spectroscopy experiments. Further, in plants, one method by which phytochelatin synthetase activity is shown is by collecting vacuoles in plant cells that store the heavy metals. Similar experiments can be conducted in bacteria.

Sodium dodecyl sulfate-polyacrylamide gel electrophoresis (SDS-PAGE). The proteins were separated in a 10% polyacrylamide gel at 200 volts constant voltage for one hour. The gel was transferred to nitrocellulose using semi-dry transfer apparatus at 60 mA constant current. The relevant proteins with His Tag were identified in a Western Blot according to manufacturer's protocol.

Protein expression and purification. The gene for the M. degradans phytochelatin synthetase was cloned into the expression vector pETBlue2. As noted earlier, the protein is expressed in E. coli and is well tolerated and highly expressed. A 50-mL culture of transformants carrying the clone of interest was grown at 37° C. to a optical density at 600 nm of 0.5 to 0.6, induced with isopropyl-β-D-thiogalactopyranoside (IPTG), and grown for an additional 3 hours at 37° C. Cells were harvested and resuspended in lysis buffer, and clarified lysates were prepared. The protein expression was monitored by virtue of the HexaHis tag that was fused to the carboxy terminus of the protein. Fusion proteins were eluted with imidazole and maltose solutions, respectively. Fractions of interest were concentrated using centrifugal concentrators with 10-kDa cutoff filters, aliquoted, and stored at 80° C.

DNA and protein sequence manipulations and analyses. Protein modules and domains were identified using the Simple Modular Architecture Tool (SMART) and pFAM database (www.smart.embl-heidelberg.de). Similarity searches were performed using the BLAST algorithm at the National Center for Biotechnology Information (NCBI) server (www.ncbi.nih.nlm.gov). Type II secretion signals were identified using the IPSORT program (www.hypothesiscreator.net/iPSORT) and the SignalP version 1.1 program (www.cbs.dtu.dk/services/SignalP). Multiple-sequence alignments were performed using the ClustalW program (www.searchlauncher.bcm.tmc.edu). Estimated protein molecular masses were calculated using the Peptide Mass Tool at the ExPASy server of the Swiss Institute of Bioinformatics (www.us.expasy.org).

Phytochelatin synthetase activity. In order to demonstrate that the M. degradans phytochelatin synthetase functions to produce phytochelatin and sequester heavy metal ions, several tests can be performed:

### EXAMPLE 1

M. degradans can be grown in the presence of heavy metals. The biomass from the culture can be collected, mineralized, and analyzed using, for example, atomic absorption mass spectroscopy. The relative amount of various heavy metals chelated by the organism can then be determined. To correlate this sequestration with phytochelatin synthetase expression, Northern Blots or RT-PCR can be used to monitor the expression of the phytochelatin synthetase gene under various conditions (e.g., the presence of heavy metals vs. no heavy metals).

### EXAMPLE 2

E. coli expressing the phytochelatin synthetase gene can be compared with wild type E. coli after each is grown in the presence of a heavy metal. The biomass from each culture would be collected and analyzed as above. E. coli (pPCSynthetaseBlue) would chelate a large amount of the heavy metals present in the culture. These tests would be run with as many heavy metal species as feasible.

It is to be understood that while the invention has been described above using specific embodiments, the description and examples are intended to illustrate the structural and functional principles of the present invention and are not intended to limit the scope of the invention. On the contrary, the present invention is intended to encompass all modifications, alterations, and substitutions within the spirit and scope of the appended claims.

1. An isolated polynucleotide comprising the nucleotide sequence of SEQ ID NO:1.

2. An isolated polynucleotide encoding a polypeptide comprising the amino acid sequence of SEQ ID NO:2.

2. An isolated polypeptide comprising the amino acid sequence of SEQ ID NO:2.

3. An isolated polynucleotide complementary to SEQ ID NO:1 under a stringency condition of from 1×SSC to 10×SSC.

4. A chimeric gene comprising at least one polynucleotide encoding a polypeptide comprising the amino acid sequence of SEQ ID NO:2.

5. The chimeric gene of claim 4, wherein the at least one polynucleotide is SEQ ID NO:1 and wherein the gene is operably linked to regulatory sequences that allow the expression of the amino acid sequence in a host cell.

6. The chimeric gene of claim 4 contained in a host cell.

7. The chimeric gene of claim 6, wherein the host cell is an Escherichia coli cell.

8. A vector comprising the chimeric gene of claim 4.

9. A vector comprising SEQ ID NO:1.

10. An isolated polypeptide expressed in Escherichia coli coding for a polypeptide having phytochelatin synthetase activity.

11. A method for isolating at least one metal from a mixture, the method comprising:
a) exposing at least one bacterium expressing phytochelatin synthetase activity to the mixture;
b) allowing the at least one bacterium to sequester the at least one metal;
c) isolating the at least one bacterium from the mixture, and
d) optionally isolating the at least one metal from the at least one bacterium.

12. The method of claim 11, wherein the at least one bacterium is selected from M. degradans or E. coli.

13. The method of claim 11, wherein the at least one metal is selected from Ag, Au, Cd, Cu, Hg, Mo, Ni, Pb, Pd, Pt, Sn, or Ti.

14. A method for remediating an area comprising at least one metal, the method comprising:
a) treating the area with at least one bacterium expressing phytochelatin synthetase activity;
b) allowing the at least one bacterium to sequester the at least one metal;
c) isolating the at least one bacterium from the area, and
d) optionally isolating the at least one metal from the at least one bacterium.

15. The method of claim 14, wherein the at least one bacterium comprises a polynucleotide further comprising SEQ ID NO:1.

16. The method of claim 14, wherein the at least one bacterium expresses a polypeptide comprising SEQ ID NO:2.

17. An apparatus for the extraction of at least one metal, the apparatus comprising:
a) at least one vessel comprising an organism able to express phytochelatin synthetase;
b) an inlet for a first composition comprising the at least one metal; and
c) a first outlet for a second composition not comprising the at least one metal.

18. The apparatus of claim 17, further comprising a second outlet for a third composition comprising the at least one metal.

19. The apparatus of claim 17, wherein the at least one organism is selected from one of M. degradans and E. coli.

20. The apparatus of claim 17, wherein the at least one metal is selected from Ag, Au, Cd, Cu, Hg, Mo, Ni, Pb, Pd, Pt, Sn, or Ti.

21. A method for identifying at least one nucleotide sequence encoding a polypeptide having phytochelatin synthetase activity from M. degradans, the method comprising constructing an M. degradans genomic library in E. coli and screening the library for phytochelatin synthetase activity.
